(19) United States
(12) Patent Application Publication
(10) Pub. No.: US 2010/0068253 A1
(43) Pub. Date: Mar. 18, 2010

(54) BLEOMYCIN PREPARATION FOR USE AGAINST SKIN TUMOURS

(76) Inventor: Sunil Chopra, London (GB)

Correspondence Address: Iandiorio Teska & Coleman, 260 Bear Hill Road, Waltham, MA, 02451-1018, US

(21) Appl. No.: 12/312,526

(22) PCT Filed: Nov. 26, 2007
(86) PCT No.: PCT/GB2007/004517

§ 371 Date: Oct. 24, 2009

(30) Foreign Application Priority Data
Nov. 28, 2006 (GB) 0623768.9

(51) Int. Cl. A61K 38/14; A61P 35/00; A61K 9/127

(52) U.S. Cl. 424/450; 514/8

(57) ABSTRACT

A preparation for use against skin tumours, which preparation has a semi-solid consistency for enabling the preparation to be applied to and remain on a patient's skin during treatment of a tumour on the patient's skin; which preparation comprises bleomycin which acts against the tumour, an elastic liposome which entraps the bleomycin; and a bleomycin containing carrier which contains the bleomycin in the elastic liposome; and which preparation is such that: (i) the concentration of the bleomycin in the bleomycin containing carrier is not less than the concentration of the bleomycin in the elastic liposome; (ii) the concentration of the bleomycin in the bleomycin containing carrier is not so great as to cause the bleomycin in the bleomycin containing carrier to leak into the elastic liposome and cause the elastic liposome to burst; and (iii) the external concentration of the bleomycin in the bleomycin containing carrier is such that it maintains an osmotic balance between internal and external environments of the elastic liposome whereby the internal concentration of the bleomycin in the elastic liposome is kept constant.

This invention relates to preparation for use against skin tumours and, more especially, this invention relates to a bleomycin preparation for use against skin tumours.

GB-A-2398495 discloses a drug delivery preparation for use against skin tumours. A preferred drug is bleomycin. With the bleomycin preparation disclosed in GB-A-2398495, two related problems have been found to occur. The first problem is providing the bleomycin preparation in a form in which it will remain on the patient's skin for a sufficient period of time to allow the bleomycin in the bleomycin preparation to take effect. For example if the bleomycin preparation is a liquid, then the liquid tends to simply run off the patient's skin and not remain on the patient's skin for the required period of time. The second problem is in providing a bleomycin preparation with a shelf life which is sufficiently long for commercial use requirements.

It is an aim of the present invention to reduce the above mentioned problems.

Accordingly, in one non-limiting embodiment of the present invention there is provided a preparation for use against skin tumours, which preparation has a semi-solid consistency for enabling the preparation to be applied to and remain on a patient's skin during treatment of a tumour on the patient's skin; which preparation comprises bleomycin which acts against the tumour, an elastic liposome which entraps the bleomycin; and a bleomycin containing carrier which contains the bleomycin in the elastic liposome; and which preparation is such that:
(i) the concentration of the bleomycin in the bleomycin containing carrier is not less than the concentration of the bleomycin in the elastic liposome;
(ii) the concentration of the bleomycin in the bleomycin containing carrier is not so great as to cause the bleomycin in the bleomycin containing carrier to leak into the elastic liposome and cause the elastic liposome to burst; and
(iii) the external concentration of the bleomycin in the bleomycin containing carrier is such that it maintains an osmotic balance between internal and external environments of the elastic liposome whereby the internal concentration of the bleomycin in the elastic liposome is kept constant.

The preparation of the present invention is such that its semi-solid consistency enables the preparation to be applied to and remain on the patient's skin for the bleomycin to take effect against the tumour. The preparation has the required shelf life for commercial use due to the above mentioned bleomycin concentrations. For example, the bleomycin preparation of the present invention may have a shelf life of up to one year as compared with a shelf life of 30-40 days of a liquid bleomycin preparation prepared in accordance with the teachings of GB-A-2398495.

The bleomycin containing carrier is preferably present in a concentration of 0.1-100 mg/ml.

Preferably, the bleomycin containing carrier is a bleomycin solution. Other bleomycin carriers may be employed, for example hyaluronic acid, an aqueous cream such as carbapol, or cellulose derivatives.

The bleomycin solution preferably comprises bleomycin in phosphate buffered saline. Other liquids may be employed.

The preparation may be in the form of a cream, ointment, gel or paste.

The bleomycin may be active bleomycin A2 and B2. Alternatively, the bleomycin may be active bleomycin A2. Alternatively the bleomycin may be active bleomycin B2.

The preparation of the present invention may be used for treatment of malignant skin cancers, vulval intraepithelial neoplasia, vulval squamous cell carcinoma, actinic keratoses, keratoacanthomas, kaposi sarcoma, Bowen's disease, and all benign tumours of viral aetiology such for example as human papilloma virus, herpes simplex virus type 8, and molluscum contagiosum.

An embodiment of the invention will now be described solely by way of example and with reference to the accompanying drawings and the following Example.

In the accompanying drawings:

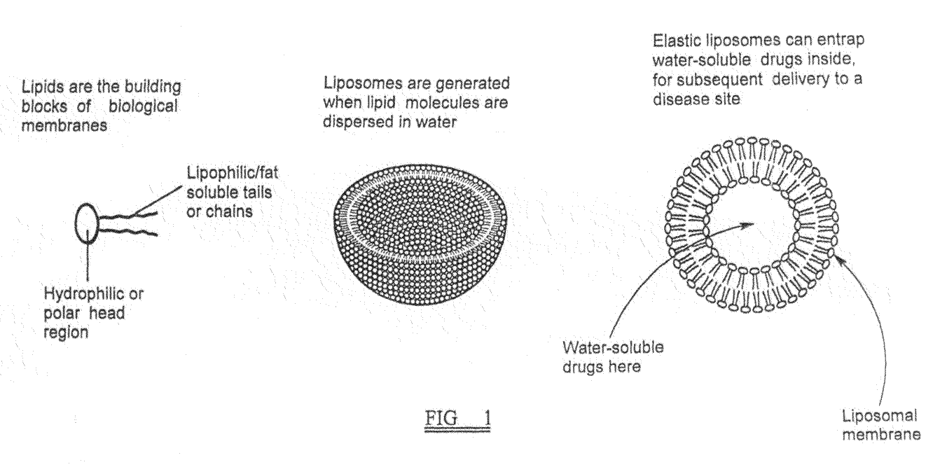

FIG. 1 shows pictorially how the elastic liposome entraps the bleomycin;

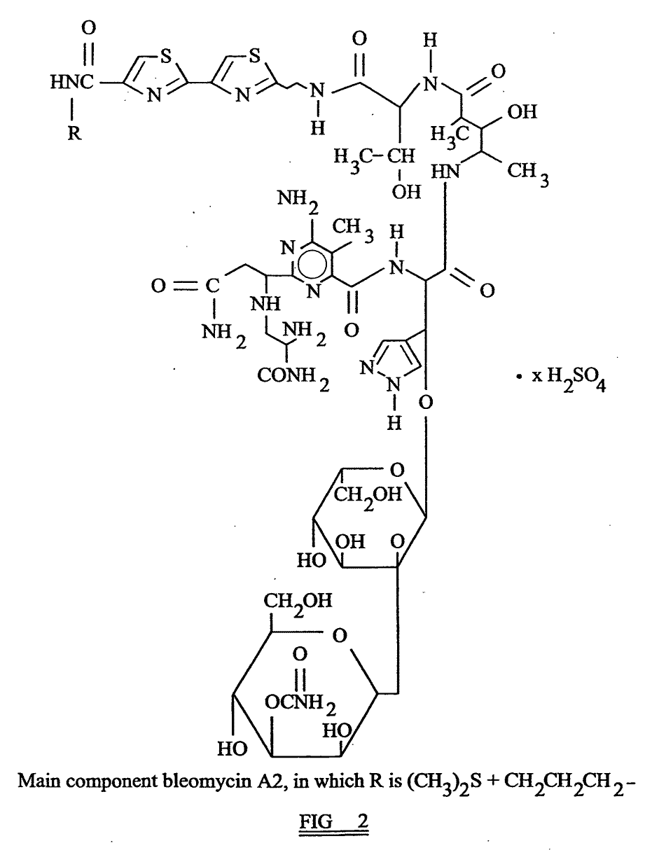

FIG. 2 gives the formula for active bleomycin A2 sulphate;

FIG. 3 shows pictorially bleomycin suspended in phosphate buffered saline;

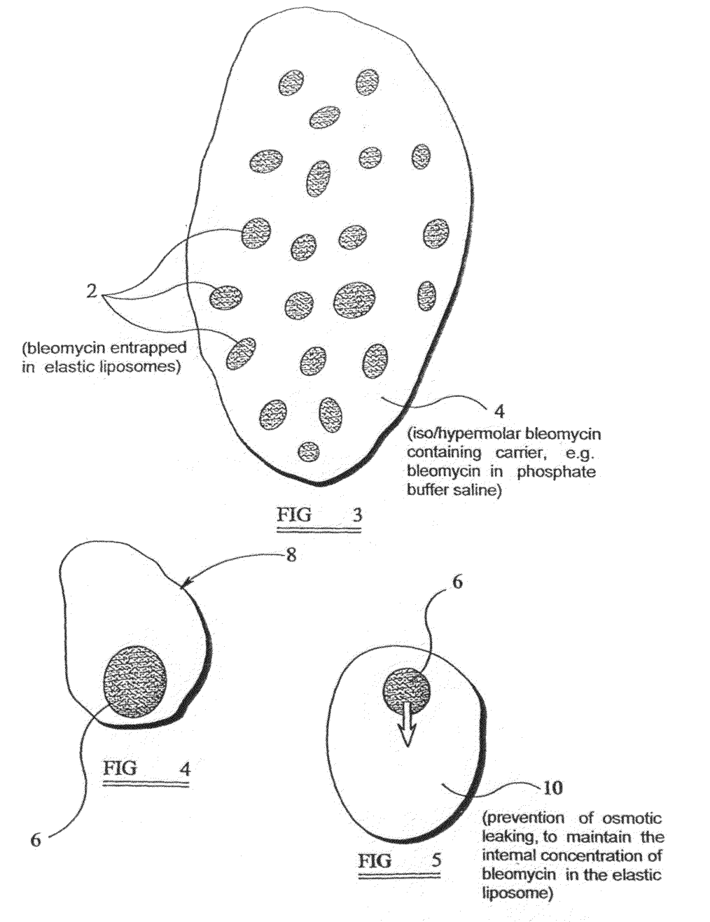

FIG. 4 shows the bleomycin in a concentrated form achieved by spinning the phosphate buffered saline of FIG. 3 to remove the phosphate buffered saline and thereby increase the concentration of the bleomycin; and

FIG. 5 shows how bleomycin at high concentrations in the form shown in FIG. 4 will leak out into a surrounding carrier by osmosis.

Referring to FIG. 1, there is shown a pictorial representation of the action of an elastic liposome in entrapping bleomycin. As can be seen from FIG. 1, lipids are the building blocks of biological membranes. Liposomes are generated when lipid molecules are dispersed in water. The liposomes entrap the bleomycin for subsequent delivery to a particular tumour site in the patient's skin. Also shown in FIG. 1 is the liposomal membrane, and water-soluble drugs trapped within it.

FIG. 2 shows the chemical structure for active bleomycin A2 where xH₂SO₄ represents the sulphate salt of the drug.

FIG. 3 shows bleomycin 2 in a suspension medium of phosphate buffered saline 4. If the bleomycin 2 in the phosphate buffered saline 4 is centrifuged, then much of the phosphate buffered saline is removed and the bleomycin 2 takes the form of a relatively large bleomycin pellet 6. The entire product 8 is then in the form of a solid paste ball. In the form shown in FIG. 4, the bleomycin will keep leaking out of the liposome (not shown) that entraps it. This leaking is shown in FIG. 5 and it is due to the bleomycin 6 being at a high concentration with respect to the carrier 10. The bleomycin in the elastic liposome leaks into the external carrier by osmosis. There is thus created an iso/hypermolar bleomycin containing carrier to maintain the internal concentration of the bleomycin in the elastic liposome.

### EXAMPLE I

A bleomycin preparation was prepared. The bleomycin preparation was a preparation for use against skin tumours. The bleomycin preparation was such that it had a semi-solid consistency for enabling the preparation to be applied to and remain on a patient's skin during treatment of a tumour on the patient's skin. The preparation comprised bleomycin which acts against the tumour, an elastic liposome which entraps the bleomycin; and a Neomycin containing carrier which contains the bleomycin and the elastic liposome. The preparation was such that:
(i) the concentration of the bleomycin in the bleomycin containing carrier is not less than the concentration of the bleomycin in the elastic liposome;
(ii) the concentration of the bleomycin in the bleomycin containing carrier is not so great as to cause the bleomycin in the bleomycin containing carrier to leak into the elastic liposome and cause the elastic liposome to burst; and
(iii) the external concentration of the bleomycin in the bleomycin containing carrier is such that it maintains an osmotic balance between internal and external environments of the elastic liposome whereby the internal concentration of the bleomycin in the elastic liposome is kept constant.

The bleomycin preparation was in the form of a gel. The gel was formed by dissolving the salt of hyaluronic acid or other gelling agents with a equi/hyperosmotic bleomycin solution. The gel was found to have an extended shelf life. The extended shelf life may be up to one year.

The gel was used to treat a patient having skin cancer. The gel was applied twice a day for four weeks. At the end of the treatment, the skin cancer had disappeared.

### EXAMPLE II

A bleomycin preparation was prepared for use against skin tumors. The belomycin preparation was such that it had a semi-solid consistency for enabling the preparation to be applied to and remain on the patient's skin during treatment of the tumor on the patient's skin. The preparation comprised belomycin which acts against the tumor, an elastic liposome which entraps the bleomycin, and a bleomycin containing carrier which contains the bleomycin and the elastic liposome. The preparation was such that:
(i) the concentration of the bleomycin in the bleomycin containing carrier is not less than the concentration of the bleomycin in the elastic liposome;
(ii) the concentration of the bleomycin in the bleomycin containing carrier is not so great as to cause the bleomycin in the bleomycin containing carrier to leak into the elastic liposome and cause the elastic liposome to burst; and
(iii) the external concentration of the bleomycin in the bleomycin containing carrier is such that it maintains an osmotic balance between internal and external environments of the elastic liposome whereby the internal concentration of the bleomycin in the elastic liposome is kept constant.

The bleomycin preparation was in the form of a cream/paste. The cream/paste was found to have an extended shelf life. The extended shelf life may be up to one year.

The cream/paste was used to treat a patient having skin cancer. The cream/paste was applied twice a day for four weeks. At the end of the treatment, the skin cancer had disappeared.

1. A preparation for use against skin tumours, which preparation has a semi-solid consistency for enabling the preparation to be applied to and remain on a patient's skin during treatment of a tumour on the patient's skin; which preparation comprises bleomycin which acts against the tumour, an elastic liposome which entraps the bleomycin; and a bleomycin containing carrier which contains the bleomycin in the elastic liposome; and which preparation is such that:
(i) the concentration of the bleomycin in the bleomycin containing carrier is not less than the concentration of the bleomycin in the elastic liposome;
(ii) the concentration of the bleomycin in the bleomycin containing carrier is not so great as to cause the bleomycin in the bleomycin containing carrier to leak into the elastic liposome and cause the elastic liposome to burst; and
(iii) the external concentration of the bleomycin in the bleomycin containing carrier is such that it maintains an osmotic balance between internal and external environments of the elastic liposome whereby the internal concentration of the bleomycin the elastic liposome is kept constant.

2. A preparation according to claim 1 in which the bleomycin containing carrier is present in a concentration of 0.1-100 mg/ml.

3. A preparation according to claim 1 in which the bleomycin container carrier is a bleomycin solution.

4. A preparation according to claim 3 in which the bleomycin solution comprises bleomycin in a phosphate buffered saline.

5. A preparation according to claim 1 and in the form of a cream, ointment, gel or paste.

6. A preparation according to claim 1 in which the bleomycin is active bleomycin A2 and B2.

7. A preparation according to claim 1 in which the bleomycin is active bleomycin A2.

8. A preparation according to claim 1 in which the bleomycin is active bleomycin B2.
